(19) United States
(12) Patent Application Publication
(10) Pub. No.: US 2010/0241145 A1
(43) Pub. Date: Sep. 23, 2010

(54) HERNIA MESH SYSTEM WITH REMOVABLE MEMORY WIRE

(76) Inventor: Douglas Wesley Cook, Exeter, CA (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/408,275

(22) Filed: Mar. 20, 2009

(51) Int. Cl. A61B 17/08; A61B 17/04

(52) U.S. Cl. 606/151; 606/228

(57) ABSTRACT

Methods and apparatuses described for hernia repair include a mesh patch with a retractable perimeter wire and dissolvable raised tabs for suturing the patch to a patient and a tool for insertion of the mesh patch into the patient.

### FIELD

Embodiments of the present invention relate generally to the field of surgical repair of abdominal wall hernias using a mesh patch.

### BACKGROUND

Repair of abdominal wall hernias is one of the most frequent surgeries performed today. Surgically implantable mesh patches are often used for the repair of these hernias and intended for permanent placement within a patient's body space. A mesh patch implanted in the patient to provide structural strength and minimize the chance of hernia recurrence. An example of such a hernia mesh patch is described in U.S. Pat. No. 6,120,539, issued to Eldridge et al.

Hernia repair utilizing mesh patches, however, typically presents at least the following difficulties. First, mesh patches typically do not have enough memory in the mesh material to easily return to a planar position once the mesh patch is inserted within the abdominal cavity. This makes accurate placement of the mesh patch difficult, as it will not easily hold its shape once inserted. Second, mesh patches typically lack points to secure sutures that would keep the surgeon from injuring structures under the mesh during fixation. This aggravates the problems in accurately and evenly placing the mesh. These issues encountered during the installation of patches often leads to non-uniform and unequal attachment of the patch to the abdominal wall around the hernia, which subsequently leads to complications for the patient and can be life-threatening.

Modifications have been made to the mesh hernia patches in an attempt to address these issues. For example, Davol Inc. of Cranston, R.I., offers a mesh with a “memory ring” that has been developed to address the first issue. The memory ring is a mono-filament that is contained in the perimeter of the patch. The mono-filament has sufficient spring tension and memory to keep the mesh planar once in place. This memory ring, however, is bulky and tends to not work well. Further, it leads to additional foreign material being placed in the patient, which can possibly lead to infection and patient rejection of the patch. There have also been multiple patient complications and one known death related to a memory ring of this type breaking after placement within the patient.

### SUMMARY

A method and apparatus are described for the repair of abdominal wall hernias that employs a mesh patch with a retractable perimeter wire. For one embodiment, the patch employs a retractable perimeter wire with a single end exposed. For an alternate embodiment, the patch employs a retractable perimeter wire with both ends exposed. For one embodiment, the perimeter wire and the planarity of the mesh patch is manipulated directly by the surgeon's hand. For an alternate embodiment, the perimeter wire, and subsequently the planarity of the mesh patch, is controlled by a hand-operated manipulator tool. A further embodiment includes raised tabs about the perimeter of the patch for suturing to the abdominal wall. For one embodiment, the tabs are made of materials such that the tabs dissolve in situ during a period of several months.

Other features and advantages of embodiments of the present invention will be apparent from the accompanying drawings and from the detailed description that follows.

### BRIEF DESCRIPTION OF THE DRAWINGS

Embodiments of the present invention are illustrated by way of example and not limitation in the figures of the accompanying drawings, in which like references indicate similar elements, and in which:

FIG. 1 shows a top-view of hernia mesh patch with a retractable perimeter wire and suture tabs according to one embodiment.

FIG. 2 shows a side view of the mesh patch with a retractable perimeter wire and suture tabs.

FIG. 3 shows a side view of a hernia mesh patch with a retractable perimeter wire and suture tabs that has been collapsed in preparation for insertion into a patient.

FIG. 4 is a flow chart of an exemplary method of repairing a hernia with a mesh patch.

FIG. 5 shows a top view of a hernia mesh patch with a retractable perimeter wire and suture tabs according to an alternate embodiment.

FIG. 6 shows a side view of a hand-operated manipulator tool for insertion and manipulation of the mesh patch.

FIG. 7 shows a side view of a hand-operated manipulator tool for insertion and manipulation of the mesh patch with the plunger extended in a fashion to accept the ends of the retractable perimeter wire.

FIG. 8 shows a side view of a hand-operated manipulator tool with the mesh patch attached and the plunger retracted in preparation for insertion into a patient.

FIG. 9 is a flow chart of an exemplary method of repairing a hernia with a mesh patch in combination with a manipulator tool.

### DETAILED DESCRIPTION

A method and apparatus are described for repairing abdominal hernias that provide for faster, more accurate insertion of a mesh patch and a reduction in patient complications. For one embodiment, a mesh patch includes a retractable stiffening perimeter wire and dissolvable raised tabs for attaching the mesh patch to the abdominal wall. For one embodiment, a hand-operated manipulator tool is used to compress, insert, and decompress a mesh patch that includes a retractable stiffening perimeter wire and dissolvable raised suture tabs.

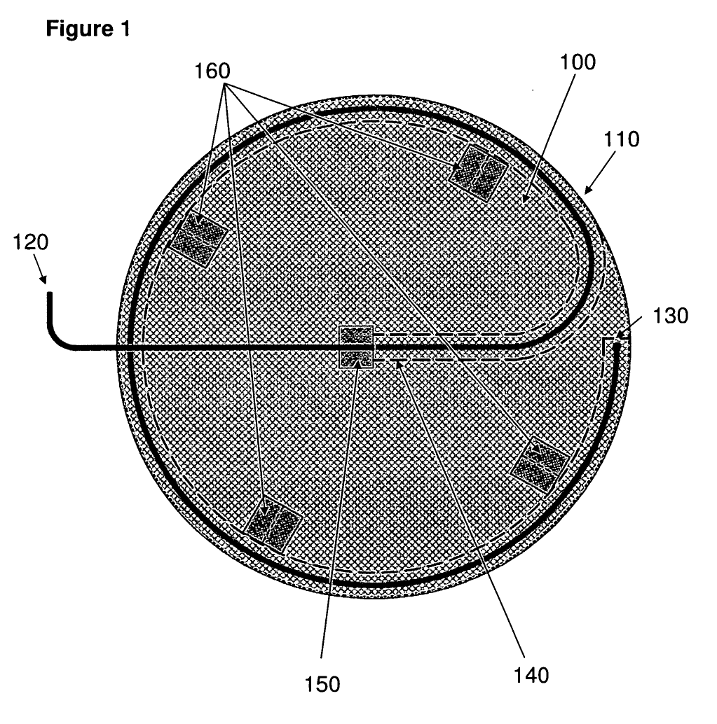

FIG. 1 illustrates a circular hernia repair mesh patch 100 including an embodiment of the invention. The mesh patch 100 is made of a permeable material, which is known in the medical industry. For one embodiment, the mesh patch 100 includes a tunnel 110 that begins in the center of the mesh patch 100 at a center tunnel opening 140. Alternatively, the tunnel 110 begins at another location on the patch that will be easily accessible during surgery for manipulation and removal of the stiffening wire 120. The tunnel 110 runs from the center of the mesh patch 100 and subsequently traverses the perimeter or near the perimeter of the mesh patch 100. The tunnel 110 terminates at the tunnel perimeter termination point 130, which is near the point at which the tunnel 110 begins to traverse the perimeter of the mesh patch 100. This provides a nearly complete circumnavigation of the mesh patch 100. For one embodiment, the tunnel 110 is formed by overlaying a first mesh with a second mesh and stitching the opposing sides of the second mesh and termination point 130 to the first mesh.

In the center of the mesh patch 100 is a manipulation tab 150 that provides a point for the surgeon to grasp the mesh patch 100 during insertion of the mesh patch 100 into the patient. A removable stiffening wire 120 is inserted within the tunnel 110. For one embodiment the stiffening wire 120 is a braided stainless steel cable. For an alternate embodiment, the stiffening wire 120 is a nylon monofilament, plastic, or other flexible wire that has a “memory” characteristic to return the stiffening wire 120 to an original position.

When the mesh patch 100 is planar, the stiffening wire 120 traverses the circumference of the mesh patch 100, and further extends from the tunnel 110 by a short distance—e.g., two to three inches. For one embodiment, the stiffening wire 120 is bent at its external end (e.g., at a 90 degree angle), providing a means for the surgeon to grasp and manipulate the stiffening wire 120. Alternatively, the external end of the stiffening wire 120 may include a small loop, ball-shaped end, or other means for the surgeon to grasp and manipulate the stiffening wire 120.

For one embodiment, one or more suture tabs 160 are affixed to the top of the mesh patch 100. The suture tabs 160 provide points by which the surgeon can affix the mesh patch 100 to the patient. The suturing of the suture tabs 160 anchors the mesh patch 100 while the patient recovers. Both the suture tabs 160 and the sutures themselves are made of materials that dissolve in situ within a period of time (e.g., a plurality of months).

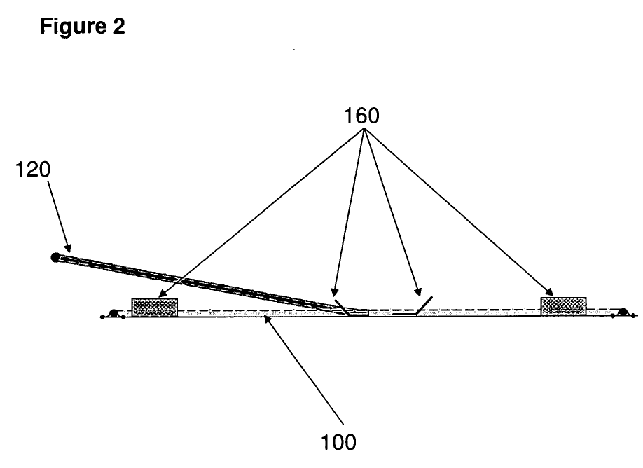

FIG. 2 is a side view of the mesh patch 100. A portion of each suture tab 160 is fixed to the planar surface of the mesh patch 100. An unfixed portion of each suture tab 160 is then folded upward to be available for affixing to the patient with a suture. As a result, the surgeon can suture the tab 160 to the patient without having to drive the suture through the bottom of the mesh. This avoids unintended punctures and associated problems.

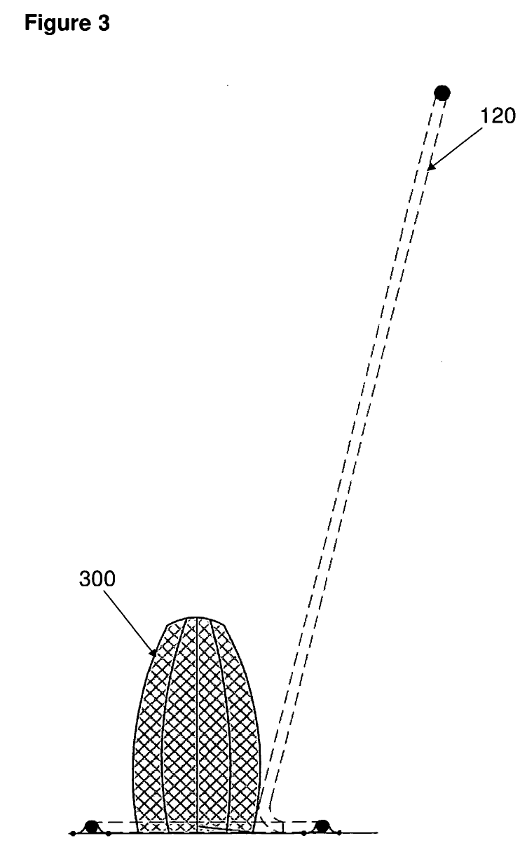

FIG. 3 shows the mesh patch 100 of FIG. 1 as it is prepared for insertion into a patient. For one embodiment, the circumference of the mesh patch 100 is smaller when compressed causing the stiffening wire 120 to protrude from the tunnel 140. The tunnel 140, when the mesh patch 100 is compressed, may now be positioned on the side of compressed mesh patch 300. Alternatively, the mesh patch 100 may be compressed or folded in a manner that does not substantially change the circumference of the tunnel 140 or otherwise cause the stiffening wire to further protrude from the tunnel 140.

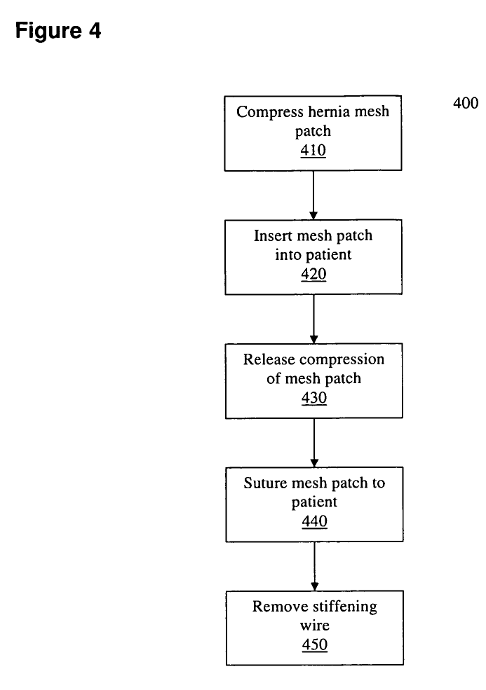

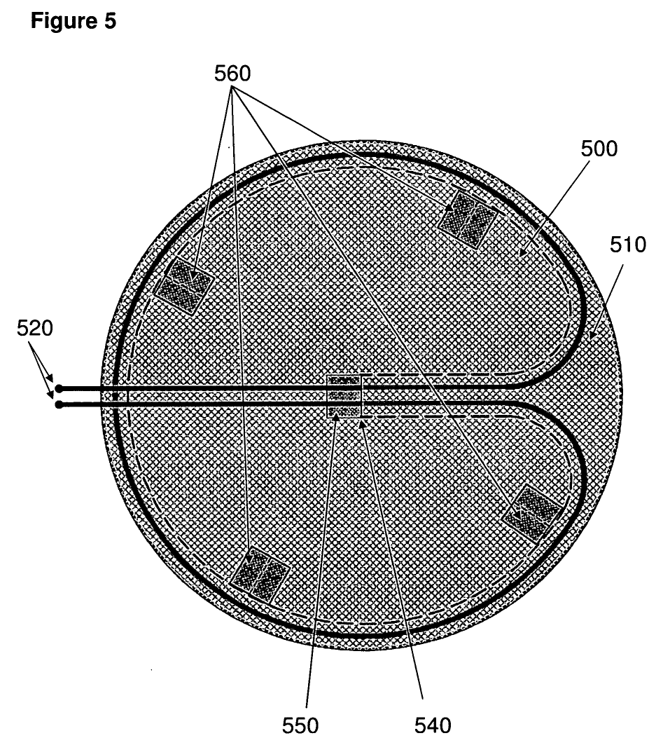

FIG. 4 is a flow chart of an exemplary method 400 of repairing a hernia with a mesh patch as described in FIGS. 1-3. At block 410, the surgeon then grasps the mesh patch 100 and compresses it. For one embodiment, the mesh patch 100 is compressed by hand. Alternatively, the mesh patch 100 is compressed by a tool, an example of which is provided below with reference to FIGS. 5-9.

At block 420, the surgeon inserts the compressed mesh patch 300 through an opening in the patient. To insert the mesh patch 100, the surgeon first dissects an opening in the patient through which the mesh patch 100 will be placed. The mesh patch 100, in a compressed shape 300, is reduced in diameter sufficiently to permit it to be inserted through a small opening in the patient and subsequently behind the abdominal wall (or other surgical location).

At block 430, the compression of the mesh patch 100 is released, allowing the stiffening wire 120 to return the mesh patch 100 to a planar state. For one embodiment, the surgeon may push a portion of the stiffening wire 120 into the tunnel 140, while also grasping the manipulation tab 150, to help return the mesh patch 100 to a planar state. At block 440, once the mesh patch 100 is centered under the hernia defect, the surgeon sutures the mesh patch 100 in place using the suture tabs 160. For one embodiment, manipulation tab 150 is used to position the mesh patch 100.

At block 450, after the mesh patch 100 is anchored in place with the sutures, the surgeon grasps the stiffening wire 120 and removes it from the mesh patch 100 through the tunnel opening 140. By removing the stiffening wire 120, less foreign material is left in the patient, which greatly reduces the chance for an infection or rejection of the hernia patch. Also, removal of the stiffening wire 120 allows the mesh patch 100 to return to its natural state: flat and flexible. This also allows the mesh patch 100 to conform to the patient's natural contours.

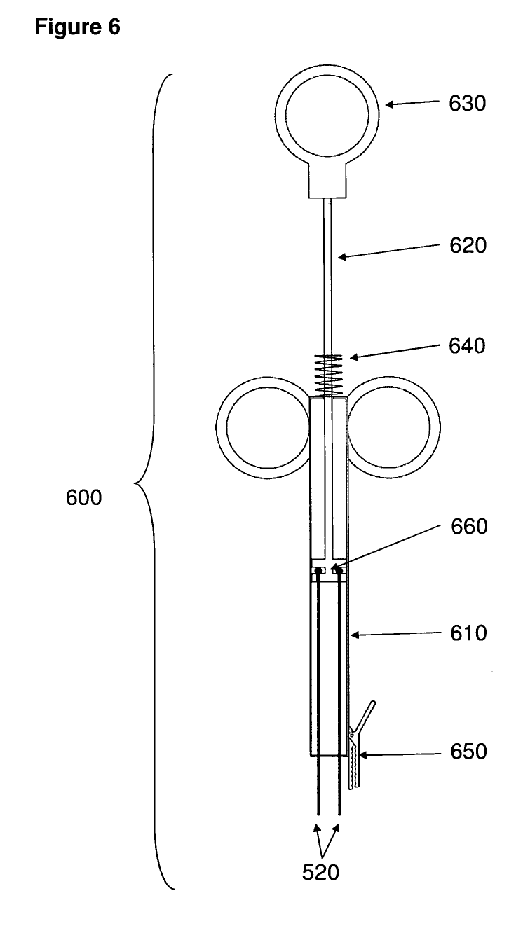

FIG. 5 illustrates an embodiment of the invention that employs a stiffening wire 520 with both ends exposed. For one embodiment, mesh patch 500 is used in conjunction with a hand-operated manipulator tool 600 which is shown in FIG. 6 and described below. Alternatively, mesh patch 500 is manipulated by hand or by another tool. The manipulator tool controls the stiffening wire to expand and contract the perimeter dimension and planarity of the mesh patch 500.

For one embodiment, the mesh patch tunnel 510 begins and ends in the center of the mesh patch 500. For one embodiment, the stiffening wire 520 enters the center tunnel opening 540, circumnavigates the mesh patch 500 perimeter or near the perimeter through the tunnel 510, and exits through the center tunnel opening 540. Alternatively, the stiffening wire 520 enters and exits separate tunnel openings. Manipulation tab 550 and suture tabs 560 are similar to manipulation tab 150 and suture tabs 160 described above with reference to FIGS. 1 and 2.

Both ends of the stiffening wire 520 are available to be grasped, e.g., by the manipulator tool 600. For one embodiment, the stiffening wire 520 is terminated with ball-shaped ends to facilitate a firm grasp by the manipulator tool 600. Alternatively, the ends of the stiffening wire 520 are bent at an angle, include loops, or other means to facilitate a firm grasp by the manipulator tool 600, by another tool, or by hand.

FIG. 6 illustrates the hand-held manipulator tool 600. The manipulator tool 600 consists of a tube-shaped barrel 610 that has a plunger 620, which is controlled by a surgeon. One end of the plunger 620 has a ring 630 affixed to it that facilitates one of the surgeon's fingers as the surgeon moves the plunger 620 up and down. For one embodiment, the plunger 620 passes through a spring 640 at the top of the barrel 610. The opposite end of the plunger 620 is contained within the barrel 610 and moves up and down inside of the barrel in accordance with the surgeon's movements or the force of the spring 640. The end of the plunger 620 contains two slots 660 that are used to grasp the stiffening wires 520. Alternatively, the tool 600 includes hooks, clasps, or another means to connect to the stiffening wires 520. The manipulator 600 further has a clasp 650 that is used to grasp the manipulation tab 550 of the mesh patch 500.

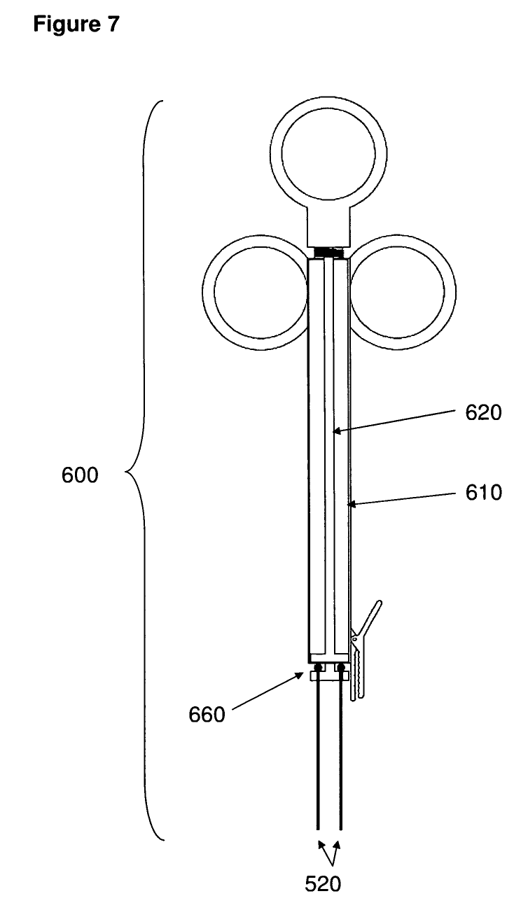

FIG. 7 illustrates the manipulator 600 with the plunger 620 firmly pushed to bottom of the barrel 610. The spring 640 is fully compressed and the slots 660 at the end of the plunger 620 are exposed. In this position, the ball-shaped ends of the stiffening wire 520 can be inserted in the slots 660, after which the plunger 620 is released, in turn pulling the stiffening wire 520 ends into the plunger 620.

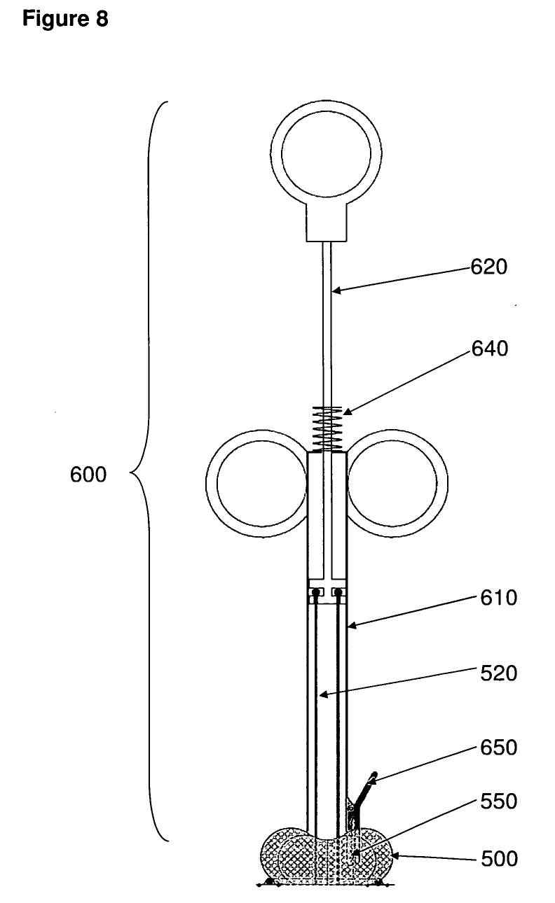

FIG. 8 illustrates the manipulator tool 600 attached to the mesh patch 500, with the stiffening wires 520 retracted into the manipulator tool barrel 610. By pulling the plunger 620 up into the housing 610 with the stiffening wires 520 attached, the perimeter of the mesh patch 500 is contracted and the mesh patch 500 takes a compressed shape.

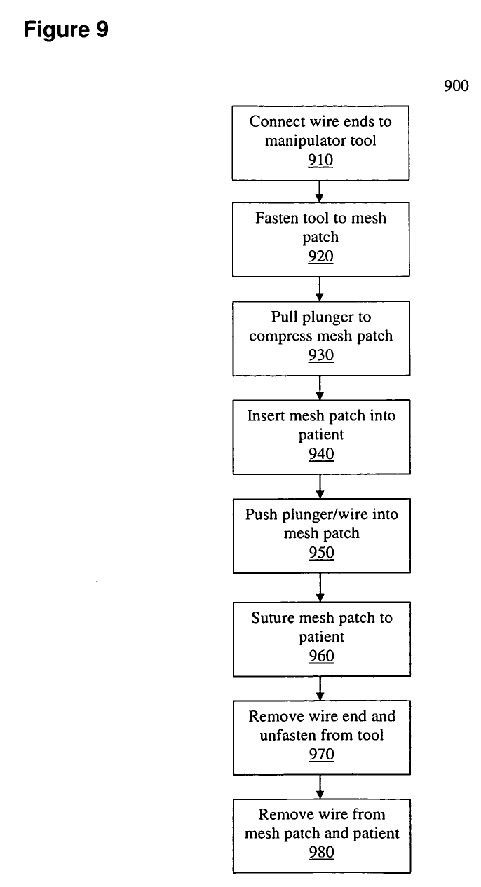

FIG. 9 is a flow chart of an exemplary method 900 of repairing a hernia with a mesh patch 500 in combination with a manipulator tool 600, as described above in reference to FIGS. 5-8. At block 910, after dissecting the opening in the patient, the surgeon prepares the mesh patch 500 for insertion by first connecting the ends of the stiffening wire 520 to the plunger 620, e.g., by inserting them into slots 660. At block 920, the surgeon then connects the clasp 650 to the mesh patch manipulation tab 550 or otherwise fastens the tool 600 to the mesh patch 500 to manipulate the positioning of the mesh patch 500 and stabilize the mesh patch 500 when the stiffening wire 520 is pulled or pushed.

At block 930, the surgeon then pulls the plunger 620 upwards. This causes the stiffening wire 520 to retract into the barrel 610, which in turn causes the perimeter of the mesh patch 500 to contract. At block 940, once the mesh patch 500 has been thus compressed in size, the surgeon inserts the mesh patch into the patient.

At block 950, the surgeon then decompresses the mesh patch 500 by pushing the plunger 620 downward. This forces the stiffening wire 520 out of the manipulator barrel 610 and into the mesh patch tunnel 510. Pushing the stiffening wire 520 into the mesh patch tunnel 510 helps the mesh patch 500 return to a planar shape within the patient. At block 960, once the mesh patch 500 is positioned, the surgeon then sutures the mesh patch 500 in place utilizing the suture tabs 560.

At block 970, the stiffening wire 520 is subsequently released from the plunger 610 by pushing the plunger 620 completely down, compressing the spring 640 which exposes the ball ends of the stiffening wire 520. The ball ends of the stiffening wire 520 are removed from the end of the plunger 620. The surgeon then releases the clasp 650. At block 980, the surgeon then removes the stiffening wire 520 from the mesh patch 500 by grasping and pulling one end of the stiffening wire 520, until the stiffening wire 520 is completely free of the mesh patch tunnel 510 and the patient.

Alternatively, only one end of the stiffening wire 520 is removed from the plunger and the tool is used to pull the other end of the stiffening wire 520 from the patch and remove it from the patient.

In the foregoing specification, the invention has been described with reference to specific exemplary embodiments thereof. It will be evident that various modifications may be made thereto without departing from the broader spirit and scope of the invention. Although for one embodiment of the invention the mesh patch is circular in shape, any shape or size mesh patch can be employed. Further, although one embodiment of the invention is used for repairing abdominal wall hernias, this invention may be applied to other hernia types. The specification and drawings are, accordingly, to be regarded in an illustrative sense rather than a restrictive sense.

What is claimed is:

1. A surgical apparatus comprising:
a mesh patch; and
a removable stiffening wire inserted within a portion of the mesh patch, wherein the stiffening wire is flexible, allowing the apparatus to be compressed, and wherein the stiffening wire causes the mesh patch to maintain a planar shape before the apparatus is compressed and after a compressing force is released.

2. The apparatus of claim 1, wherein the mesh patch includes a tunnel along a portion of the perimeter of the mesh patch and wherein the removable stiffening wire is inserted into and removable from the tunnel.

3. The apparatus of claim 2, wherein the tunnel includes an opening at the center of the mesh patch and wherein the removable stiffening wire is inserted into and removable from the opening.

4. The apparatus of claim 1, further comprising:
a dissolvable suture tab affixed to the mesh patch.

5. The apparatus of claim 1, further comprising:
a tab affixed to the mesh patch to be clasped by hand or a tool to manipulate the positioning of the mesh patch.

6. The apparatus of claim 1, wherein a portion of the removable stiffening wire remains external to the mesh patch and wherein the external portion of the removal stiffening wire is bent at an angle.

7. The apparatus of claim 1, wherein two end portions of the removable stiffening wire remain external to the mesh patch and wherein the external end portions of the removal stiffening wire include a ball-shaped ends.

8. The apparatus of claim 1, wherein the stiffening wire is a braided steel cable.

9. The apparatus of claim 1, wherein the stiffening wire is a nylon monofilament.

10. A surgical apparatus to compress a mesh patch for insertion into a patient, the apparatus comprising:
a housing;
a plunger, wherein a portion of the plunger resides and moves along a controlled path within the housing, the plunger to connect to a removable stiffening wire of a mesh patch; and
a fastener, coupled to the housing, to fasten the apparatus to the mesh patch and hold the mesh patch in place while the plunger pulls a portion of the removable stiffening wire out of the mesh patch and causes a perimeter of the mesh patch to contract.

11. The apparatus of claim 10 further comprising:
a spring to resist one direction of movement of the plunger.

12. The apparatus of claim 10, wherein the plunger connects to two opposing ends of the stiffening wire.

13. The apparatus of claim 10, wherein the plunger includes two slots to receive ball-shaped ends of the stiffening wire, wherein the slots are exposed when the plunger is at an end of its path, and wherein the apparatus firmly grasps the stiffening wire when the portion of the plunger containing the slots is inside the housing.

14. The apparatus of claim 10, wherein the fastener is a clasp to grasp a tab on the mesh patch.

15. The apparatus of claim 10 further comprising a ring coupled to the plunger or to the housing to receive a digit of a hand operating the apparatus and to assist in the movement of the plunger within the housing.

16. A method of repairing a hernia comprising:
compressing a mesh patch, wherein the mesh patch includes a removable stiffening wire inserted within a portion of the mesh patch, wherein the stiffening wire is flexible and causes the mesh patch to maintain a planar shape before the mesh patch is compressed and after a compressing force is released;
inserting the mesh patch into a patient;
releasing the mesh patch from a compressed state;
affixing the mesh patch to the patient; and
removing the stiffening wire from the patch and the patient.

17. The method of claim 16, wherein the mesh patch includes a dissolvable suture tab and wherein affixing the mesh patch to the patient includes suturing the dissolvable suture tab to the patient.

18. The method of claim 16, further comprising:
manipulating a position of the mesh patch by clasping a tab affixed to the mesh patch.

19. The method of claim 16, wherein the stiffening wire is removed by clasping a ball-shaped end or bent portion of the stiffening wire that is exposed from the mesh patch and pulling the stiffening wire from the mesh patch.

20. The method of claim 16, wherein the mesh patch is compressed by hand.

21. The method of claim 16, wherein the mesh patch is compressed by a tool.

22. The method of claim 21, wherein the compressing of the mesh patch by a tool comprises:
fastening the tool to the mesh patch to manipulate the position of the mesh patch and hold the mesh patch in place; and
pulling a portion of the stiffening wire out of the mesh patch to cause a perimeter of the mesh patch to contract.

23. The method of claim 22, wherein the tool includes a housing and a plunger that resides and moves along a controlled path within the housing, wherein the fastening and pulling further comprises:
connecting the plunger to two ends of the stiffening wire; and
pulling the plunger along the controlled path to draw the ends of the stiffening wire into the housing.

24. The method of claim 23, further comprising:
releasing an end of the stiffening wire from the plunger; and
pulling one of the ends of the stiffening wire to remove the stiffening wire from the mesh patch and the patient.

25. The method of claim 16, further comprising:
pushing a portion of the stiffening wire into the mesh patch to cause mesh patch to return to a planar shape.
